(19) United States
(12) Patent Application Publication
(10) Pub. No.: US 2024/0033494 A1
(43) Pub. Date: Feb. 1, 2024

(54) Apparatus for Accurately Positioning an Endocaval Lead

(71) Applicant: Joesph M. Bunch, Romeoville, IL (US)

(72) Inventor: Joesph M. Bunch, Romeoville, IL (US)

(21) Appl. No.: 18/377,296

(22) Filed: Oct. 5, 2023

Related U.S. Application Data
(63) Continuation-in-part of application No. 17/169,446, filed on Feb. 6, 2021.

(51) Int. Cl. A61M 39/02; A61M 25/01

(52) U.S. Cl. CPC A61M 39/0247; A61M 25/0102; A61M 2025/0166; A61M 2205/3317; A61M 2210/125; A61M 2039/0205; A61M 2039/0258; A61M 2039/0267; A61M 2039/0282; A61M 2205/0272

(57) ABSTRACT

An electrical connector clamp for attaching to a distal end of a wire of a Central Venous Access Device (CVAD) including a first plate having a first side, a second side, a first edge, and a second edge. A second plate also is included having a third side, a fourth side, a third edge, and a fourth edge, wherein the first edge and the third edge are connected by a hinge that enables the first and second plates to pivot relative to each other, and the hinge enables the first side and the third side to come in contact. A latch holds the first side of the first plate against the third side of the second plate in a closed position. An electrically conductive material is on the first side of the first plate, and an electrical connector is on the second side of the first plate that is electrically connected to the electrically conductive material on the first side of the first plate. The first and third sides are to be pivoted together in the closed position around a wire to provide an electrical connection between the wire and the electrical connector.

### CROSS-REFERENCE TO RELATED APPLICATION

This application is a Continuation-In-Part (CIP) application of application Ser. No. 17/169,446, filed on Feb. 6, 2021, which is hereby incorporated by reference.

### BACKGROUND OF THE INVENTION

### Field of the Invention

The present invention relates generally to Central Venous Access Devices (CVADs), and more particularly, to devices for properly locating the distal end of the CVAD relative to a patient's heart.

### Description of Related Art

Central Venous Access Devices (CVADs) are widely used in the medical field. Central venous system access in a patient is an important aspect of administering intravenous therapy, such as vesicants, chemotherapy, and Total Parenteral Nutrition (TPN). Some drugs are caustic or irritating to lower arm veins that have smaller diameters. The standard of choice now is to deliver medications into larger blood vessels located in the central portion of a patient's chest. A catheter is advanced into the largest vein in the body, called the Superior Vena Cava (SVC). Central lines are precisely placed at the junction of the SVC and the atrium of the heart called the Cavoatrial Junction (CAJ).

By definition, a Central Venous Access Device (CVAD) is a venous access device that ultimately terminates in the superior vena cava (SVC) or at right atrium (RA) junction, referred to as cavoatrial junction (CAJ). CVADs can be inserted centrally (centrally inserted central catheter; CICC) or peripherally (peripherally inserted central catheter; PICC). PICCs are placed through the basilic, brachial, cephalic, or medial cubital vein of the arm.

The right basilic vein is the vein of choice due to its relative larger size and location and straightest route to the axillary vein, then through the subclavian, and finally, in the SVC. The cephalic vein is another option for PICC-line placement but, in addition to being smaller than the basilic vein, the PICC course through the upper arm can be very tortuous. PICCs placed through this vein are thought to have a higher incidence of mechanical phlebitis, and the PICC's sharp angle of insertion makes it difficult to advance the catheter. The brachial vein is another option due to its larger size, however, it is smaller and runs deeper than the basilic vein. The brachial vein also passes close to the brachial artery and median nerve.

PICCs are generally ordered more frequently due to their relatively safe method for obtaining central venous access. PICCs are indicated in patients who require venous access for several weeks to months due to their low infection rates. Additionally, PICCs can be managed both in inpatient and outpatient settings. Some common indications include patients with difficult intravenous access, continuous infusion of vesicants, or hyperosmolar/extreme PH infusions and chemotherapy.

PICC lines vary in design and size, ranging from 3 feet to 6 feet in adults and 45 to 60 centimeters in length. PICCs also can be both valved or non-valved, and contain single, double or triple lumen catheters that aid in delivering medicines that are not compatible to mix. Different brands of catheters may have subtle differences in their packaging and equipment. PICCs are commonly placed by specially trained registered nurses and or physicians. However, many institutions have dedicated vascular access nurses. CVADs are routinely placed safely at the bedside using ultrasound guidance. Ultrasound guidance shows considerably improved outcomes. As with any procedure, proper preparation is essential, thus ensuring that all necessary equipment and materials are present is paramount for successful outcomes.

A sterile technique is especially vital for this procedure to decrease the risk of catheter-related bloodstream infections (CRBSIs). Education of standardized catheter placement, care, maintenance and prevention of infection have been shown to drastically reduce the incidence of CRBSIs. The Seldinger technique is by far the most commonly used method for placing PICCs. Peel-away sheaths are commonly used and require large veins to accommodate larger sized introducers, which potentially expose patients to increased risk of excessive bleeding. Additionally, peel-away catheters are known to cause air emboli if caution is not taken to minimize risk.

A peripherally inserted central venous catheter (PICC or PICC line) is initially inserted into a peripheral vein, normally in the upper arm of the patient. The catheter is then advanced through larger veins towards the heart for a prescribed distance. PICCs are intended to remain in place for extended periods, such as from a few days up to a few years. A PICC typically has one or more lumens that are externally accessible by a clinician and converge into a single catheter body that is internally implanted within a vein of the patient. The tubes are adapted to receive therapeutic agents, which are then released through a distal tip of the catheter body into the central venous system of the patient.

The most common technique used by a clinician to gain access to the central venous system of a patient with a PICC is a modified Seldinger technique. This technique involves the clinician first inserting a needle through the patient's skin at a peripheral location and into a vein to form a venotomy. The clinician then inserts a guidewire through the passageway of the needle and into the vein. Next the needle is removed from the vein, and a peelable sheath is inserted over the guidewire and into the vein. The guidewire then is removed, and then the inducer of the peelable sheath is removed. A catheter is then inserted into the peel away sheath, and then the peel away sheath is removed leaving the catheter within the patient.

Current standards for proper catheter insertion depend on the type of catheter and the treatment being provided. A stated earlier, PICC lines are commonly inserted into a brachial, cephalic or basilic vein in the arm and advanced through the venous system towards the SVC. Current medical standards recommend that the distal tip of the catheter terminate in the lower third portion of the SVC/Cavoatrial junction (CAJ), which is the junction of the SVC and the Right Atrium (RA). However, since PICCs are commonly inserted into a vein in the arm and advanced through the venous system to reach the SVC, the PICC line tip may be inadvertently malpositioned in a non-target area, such as the internal jugular, the subclavian vein, or too far past the CAJ and into the heart.

Ultrasound, chest radiographs, and fluoroscopy techniques are used by trained nurses or doctors to aid in the insertion of the catheter and to confirm that its tip is properly positioned. There are only a few centimeters of space in the superior vena cava vessel where the tip can be safely located. Catheter tip location techniques have improved the ability of medical professionals to verify the location of the catheter tip. Fluoroscopy provides the operator with real-time images of a patient's anatomy using a fluoroscope. Another technique uses a combination of an electromagnetic beacon and an electromagnetic detection element to track the beacon positioned near the catheter tip. Many techniques also have been described for using electrocardiography (ECG) to assist with catheter tip placement by measuring an ECG signal from an intravascular (IV) electrode positioned at or near the catheter tip.

Tracking ECG waveform changes measured from an IV electrode as the catheter advances through the vasculature towards the Sinoatrial Node (SA node) can provide valuable feedback to a medical professional for properly placing the catheter, since the SA node is located near the SVC-RA junction. Specifically, tracking the P-wave morphology is known to be a valuable tool. For example, as the IV electrode advances down the SVC towards the SA node, the amplitude of the P-wave will start to rise. The amplitude of the P-wave will eventually peak when the IV electrode is closest to the SA node, and eventually start to decrease in amplitude and develop a negative wave as the IV electrode moves away from the SA node and enters the RA.

In order to understand the current process for confirming the tip or distal end of a PICC is positioned at the precise desired location, there are a few basic concepts that need to be understood. An electrocardiogram is a recording of “data” of a human heart's electrical conduction system. The heart essentially has its own action potential cells in the heart muscle that rely on an “internal battery” of fibers to generate and regulate the conduction of electrical impulses or energy to generate a muscle contraction of the heart, similar to a Transcutaneous Electrical Nerve Stimulation (TENS) unit.

The starter or initiator of an electrical impulse in the heart is a node located in the right atrium, called the Sinoatrial Node (SA node). This node generates electrical impulses and has extensions that surround the atrial muscle that connects to the lower heart muscle to conduct generated electrical impulses. Electrical impulses are generated by positive (+) and negative (−) ions, depolarization/repolarization of the heart muscle, referred to as the action potential. An electrocardiogram (ECG or EKG) records the tracing or data of the electrical signals from the heart. A wave of depolarization traveling towards a positive electrode results in a positive deflection in the ECG tracing. A wave of depolarization traveling away from a positive electrode results in a negative deflection. An ECG detects those electrical impulses traveling from the upper to lower parts of the heart.

Referring to FIG. 1, bipolar recordings utilize standard limb lead configurations. Lead I has the positive electrode on the left arm (LA), and the negative electrode on the right arm (RA), and measures the potential difference between the two arms. Thus, I=LA−RA. An electrode on the right leg (RL) functions as a reference electrode for recording. In the Lead II configuration, the positive electrode is on the left leg and the negative electrode is on the right arm, thus II=LL−RA. Finally, Lead III has the positive electrode on the left leg and the negative electrode on the right arm, thus III=LL−LA. These three bipolar limb leads roughly form an equilateral triangle, with the heart at the center, which is referred to as the Einthoven's triangle in honor of Willem Einthoven who developed the electrocardiogram in the early 1900s.

If three electrodes are connected to the patient for an ECG, the negative (−) is on the right arm, the positive (+) is on the left arm, and the lead on the chest adjacent to the heart also is the positive (+) lead. With a two electrodes setup, the negative lead is on the right arm and the positive lead is on the chest adjacent to the heart. The positive lead looks toward the negative lead to record the electricity flowing from the top of the heart to the lower part. With the heart being three-dimensional, electrical impulses flow from the superior to the anterior/posterior and then to inferior and lateral regions. An ECG can record all of these signals simultaneously. However, with regard to confirming a proper location of the catheter tip, a clinician is focusing on lead II of the ECG data acquisition (II=LL−RA).

An ECG is recorded in waves, and those waves are labeled as “PQRST.” depolarization of the atria produces deflections or waves in the ECG tracing, called P-waves. Depolarization is the stimulus for contraction of the atrial muscle. Because it is so small, atrial repolarization is usually not visible on ECG. The QRS complex represents the depolarization of the ventricles, and the T wave represents the repolarization of the ventricles. When the atrial tissue reaches maximal potential and depolarizes, the atrial tissue sends an electrical impulses through the atrial tissue before the atrial muscle contacts, which is the upstroke of the “P” wave on a normal ECG. Before a muscle can contract the cardiac muscles needs to recharge.

Electrical impulses travel along the conduction pathway from the SA node throughout the entire cardiac muscle to initiate contraction and relaxation. However, the real focus of a clinician is on the P wave in which the negative right arm (RA) lead essentially is the intravascular conductive lead, wherein a conductive wire is connected to the RA lead that results in a +60 degree of orientation of Einthoven's triangle. As the wire is inserted further into the arm vein of a patient, the tip of the catheter becomes a means for conduction, and when used in conjunction with the ECG lead, the wire becomes part of the lead, functioning as an “extension cord” essentially. As the conductive wire enters the great vein of the SVC, the “P” wave will begin to rise, indicating a maximum potential. The closer the tip of the conductive wire gets to the “initiator of impulse,” or the SA node, the greater the action potentials, resulting in a maximum P wave spike. As the conductive wire passes the SA node, the wave will drop off and produce a negative waveform as the action potential is significantly less, alerting the clinician to pull the catheter back. The desired location is the Cavoatrial Junction (CAJ), which is defined as 1-2 centimeters above the atrium and 1-2 centimeters below the atrium, which is a very narrow margin.

In order for the tip of the catheter to be properly positioned using an ECG, the lead signals must be clear and have little to no interference or artifacts. Unfortunately, such problems are not uncommon. Furthermore, it takes significant time to prepare and connect ECG leads to a patient, which is a concern in the current Covid 19 virus environment, in addition to general concerns of infection during such procedures.

The Bard Access Systems Sherlock®, a PICC catheter placement system, by Bard Access Systems, of Lucent Medical Systems in Kirkland, Washington, can only be used with Bard PICC catheters. The Bard Access Systems Sherlock® comes with a preloaded stylet as the conductor median that attaches to a Y-shaped electronic sensor that is seated upon the chest of a patient. The Bard system requires significant time to set up and utilizes a sensor that does not provide a direct electrical connection between the distal end of the catheter and an ECG monitor. The Bard system also is not an inexpensive product.

Another PICC locating device is provided by Teleflex, Inc. in Morrisville, N.C., called the Arrow® VPS Rhythm® Device with optional TipTracker™ Technology. The Teleflex device is very similar the Bard device as it also utilizes a Y-shaped sensor that is seated upon the chest of a patient to properly locate the distal end of the catheter. The Teleflex device includes a module to connect to the stylet as the conduction median and multiple electrode leads. Furthermore, the chest area for the Teleflex sensor must be free from any and all cardiac pads, cables or wires, which is a big factor in a critical ill patient. Devices such and the Bard and Teleflex system must be attached to a trolley or mounting system to handle and support all the equipment and large video monitor as a display.

In addition to the concerns discussed above, finding more economical and effective ways to electrically connect the guidewire of a CVAD to an ECG waveform monitor is desirable.

Accordingly, there is a need for an improved method of accurately positioning the distal end of a catheter within a patient that produces improved ECG signals and minimized the time and equipment needed to prepare and properly position the distal end of a catheter within a patient.

### ASPECTS AND SUMMARY OF THE PRESENT INVENTION

One aspect of the present invention is to provide an apparatus and method for properly locating a distal end of a catheter within a patient that reduces the amount of needed medical equipment.

Another aspect of the present invention is to provide an apparatus and method for properly locating a distal end of a catheter within a patient that reduces the amount of time for setting up and performing the procedure.

A further aspect of the present invention is to provide an apparatus and method for properly locating a distal end of a catheter within a patient that provides improved ECG signals with no or minimal interference and no artifacts.

Another aspect of the present invention is to provide an apparatus and method for properly locating a distal end of a catheter within a patient that provides improved ECG signals with no or minimal interference and no artifacts, and the method is compatible with multiple types of devices for displaying ECC signals, including several that are relatively inexpensive.

An additional aspect of the present invention is to eliminate the exposure to radiation from a fluoroscopy by eliminating the need to use fluoroscopy to properly locate the distal end of a catheter.

A further aspect of the present invention is to improve mobility of the device by minimizing components.

Another aspect of the present invention is to reduce costs associated with properly locating a distal end of a catheter within a patient.

An additional aspect of the present invention is to enable the components of the device to be single use and disposable, thus reducing the risk of infection and eliminating cleaning costs.

In order to provide these aspects and others, the present invention provides both a method and an apparatus for utilizing an Endocaval Electrocardiogram (ECG) signal for central venous access device (CVAD) placement. The process is capable of transmitting the position of a CVAD tip and assessing its location relative to the cavoatrial junction (CAJ). The data received is interpreted by endocaval (EC) signal origin relative to the EC-electrode lead. The process is based on electrophysiology basics in which a standard ECG receives electrical impulses and generates waves that are recorded on an ECG.

In accordance with a preferred CVAD insertion method of the present invention, a desired vein is located for insertion using ultrasound, and then a needle is inserted into the vein creating a venipuncture. Next a guidewire is inserted though the needle, and then the needle is removed leaving the guidewire within the vein. A peel away sheath with a micro introducer is inserted over the guidewire and into the venipuncture, and then the guidewire is next removed. The distance from the venipuncture to the SVC is measured externally, and then a catheter with a stylet inside a lumen of the catheter is inserted into the vein inside the peel away sheath. The position of the stylet inside the catheter is premeasured and locked in place externally on the proximal end of the catheter so the stylet is positioned at the distal end of the catheter, but cannot exit the distal end of the catheter. Once the catheter is inserted to the approximate desired location based upon the external measurement, the peel away sheath is removed.

Next, in accordance with the invention to properly position the distal end of the catheter within the SVC, the Scout™ clip is attached to the proximal end of the stylet exiting the proximal end of the catheter, and the opposing end of the Scout™ electrical lead is connected to an ECG monitor. In another embodiment of the invention, the proximal end of the stylet includes a connector that directly connects to an ECG monitor. The distal end of the catheter including the distal end of the stylet is then precisely positioned within the SVC by looking for a peak in the P-wave. After the distal end of the catheter is precisely located, the stylet is removed and the catheter and disconnected from the ECG. The IV connector on the proximal end of the catheter is then secured adjacent to the venipuncture where the catheter enters the vein.

In accordance with a preferred dialysis catheter line insertion method of the present invention, a desired centralized vein is located for insertion using ultrasound, and then a needle is inserted into the jugular vein to create a venipuncture. A guidewire is inserted into the vein through the needle, and then the needle is removed. An introducer or dilator is then inserted over the guidewire and into the venipuncture in order to dilate the opening of the venipuncture, which follows the Seldinger technique. The dilator is removed, and then the catheter is inserted over the guidewire into the venipuncture, and then the guidewire is removed. The distance from the venipuncture to the SVC is measured externally, and then a catheter with a stylet inside a lumen of the catheter is inserted into the vein. The position of the stylet inside the catheter is premeasured and locked in place externally on the proximal end of the catheter so the stylet is positioned at the distal end of the catheter, but cannot exit the distal end of the catheter. The catheter is inserted to the approximate desired location based upon the external measurement.

Next, in accordance with the invention to properly position the distal end of the catheter within the SVC, similar to precisely positioning the distal end of the catheter for a PICC line, the Scout™ clip is attached to the proximal end of the stylet exiting the proximal end of the catheter, and the opposing end of the Scout™ electrical lead is connected to an ECG monitor. In another embodiment of the invention, the proximal end of the stylet includes a connector that directly connects to an ECG monitor. The distal end of the catheter including the distal end of the stylet is then precisely position within the SVC by looking for a peak in the P-wave. After the distal end of the catheter is precisely located, the stylet is removed and the catheter and disconnected from the ECG. The IV connector on the proximal end of the catheter is then secured adjacent to the venipuncture where the catheter enters the vein.

The apparatus of the present invention includes a premeasured stylet with a locking proximal end in the catheter so the distal end of the stylet is locked at the distal end of the catheter without exiting the distal end of the catheter.

The apparatus of the present invention further provides a coverplate connector enabling ECG leads to connect to a small and highly portable ECG monitor.

Prior to inserting a needle into a patient to form a venipuncture, the apparatus of the present invention has a set time of less than a minute, in contrast to conventional ECG set ups for positioning a catheter, which can take over ten minutes. Additionally, the apparatus of the present invention can be used with any CVAD device or any ECG monitor.

The present invention enables a stylet to electronically connect directly to any ECG monitor universally. The SCOUT™ lead and a stylet are preferably configured with a male snap electrode head enabling a quick and accurate data acquisition of the precise location of the distal end of a PICC, while further providing easy portability by reducing component size and minimizing the amount of time spent at a patient's bedside. The present invention also reduces the amount of equipment, wires and cables used by prior art PICC positioning systems.

The present invention further improves signal acquisition by using a direct, wired, electrical connection, rather than an indirect sensor using multiple contact points. The direct wired electrical connection of the present invention minimizes electrical artifacts associated with a wireless sensor using multiple connections and potential undesired movement common with skin contact sensors.

This invention improves efficiency in the time needed for confirmation of the correct placement of the distal end of the PICC as well as initial set up time. Moreover, the present invention can be used to confirm the accurate placement of any central venous access device such as: PICC, CVAD, dialysis catheter, tunneled CVADS, and any other device wherein the tip or distal end of the CVAD is to be accurately placed at or near the cavoatrial junction.

The present invention also is specifically designed to be the only confirmatory stylet system to function with a new novel polyvinyl alcohol PICC. Due to the increased electrical impedance produced by the catheter, the present invention is the only product known to the inventor compatible with the polyvinyl alcohol based PICC.

The SCOUT™ lead and stylet configured in accordance with the present inventor provide a significantly improved and highly accurate confirmation method for determining the position of the tip of any catheter. The present invention only needs an ECG monitor, which can be a portable ECG monitor, a bedside monitor, a smart tablet, or even a smart phone having a display screen, such as an iPhone®. A Bluetooth accessory can wirelessly connect to the smart device, such as an Android or other IOS operating device having a display screen that is small, compact, and cuts down on cable clutter and is extremely portable.

In accordance with another aspect of the present invention, an electrical connector, the SCOUT™ connector, for attaching to a distal end of a guidewire or stylet wire of a CVAD is provided having a first plate with a first side, a second side, a first edge, and a second edge. A second plate also is included having a third side, a fourth side, a third edge, and a fourth edge, wherein the first edge and the third edge are connected by a hinge that enables the first and second plates to pivot relative to each other, and the hinge enables the first side and the third side to come in contact with each other. A latch holds the first side of the first plate against the third side of the second plate in a closed position. An electrically conductive material is on the first side of the first plate, and an electrical connector is on the second side of the first plate that is electrically connected to the electrically conductive material on the first side of the first plate. The first and third sides are to be pivoted together around a wire to provide an electrical connection between the wire and the electrical connector.

The foregoing has outlined, rather broadly, the preferred features of the present invention so that those skilled in the art may better understand the detailed description of the invention that follows. Additional features of the invention will be described hereinafter that form the subject of the claims of the invention. Those skilled in the art should appreciate that they can readily use the disclosed invention and specific embodiments as a basis for designing or modifying other structures for carrying out the same purposes of the present invention, and that such other structures do not depart from the spirit and scope of the invention in its broadest form.

### BRIEF DESCRIPTION OF THE DRAWINGS

FIG. 1 is a prior art diagram of Einthoven's triangle, which provides an imaginary formation of three limb leads in a triangle used in electrocardiography;

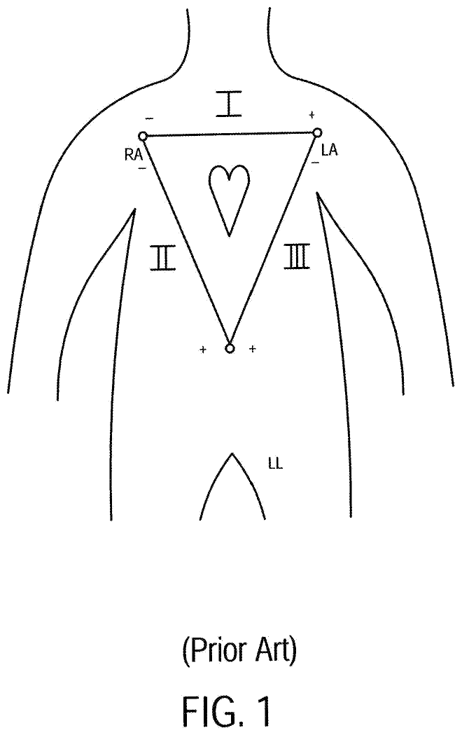

FIG. 2 is a diagram of a patient's body illustrating the insertion of a PICC in accordance with the present invention;

FIG. 3 is drawing of a stylet for a catheter configured in accordance with the presenting invention;

FIG. 4 illustrates a spring engaged ECG electrode connector configured in accordance with the present invention;

FIG. 5a is a perspective view of the bottom of an ECG lead connector coverplate for a portable ECG monitor configured in accordance with the present invention;

FIG. 5b is a perspective view of the top of the ECG lead connector coverplate shown in FIG. 5a;

FIG. 5c is a perspective view of the top of the ECG connector coverplate shown in FIGS. 5a and 5b wherein connector leads are about to be connected to the coverplate;

FIG. 6 is a flowchart of a method of inserting a PICC line into a patient in accordance with a method of the present invention;

FIG. 7 is a flowchart of a method of inserting a dialysis line into a patient in accordance with a method of the present invention;

FIG. 8 is a perspective view of the electrical connector clamp or Scout™ connector configured in accordance with the present invention and in the open position; and

FIG. 9 is a perspective view of the electrical connector clamp shown in FIG. 8 in the closed position.

### DETAILED DESCRIPTION OF THE PREFERRED EMBODIMENTS

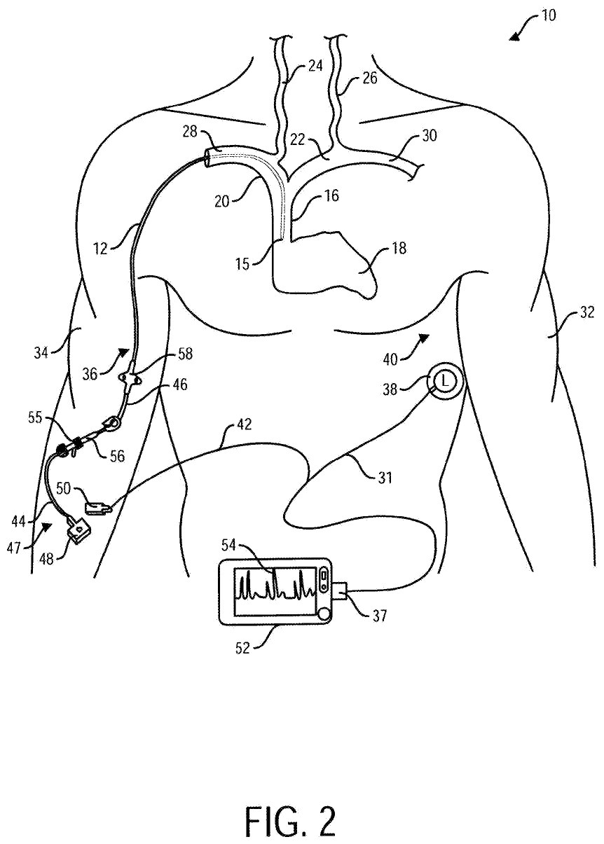

FIG. 2 illustrates the body 10 of a patient receiving a PICC 12 wherein the distal end 15 of the PICC 12 has been inserted to the proper location in the superior vena cava (SVC) 16 just above the heart 18. The SVC is formed at the junction of the right brachiocephalic 20 and the left brachiocephalic 22. Further illustrated above the heart 18 are the right jugular 24 and the left jugular 26. Also illustrated is the right subclavian 28 and the left subclavian 30. The left arm 32 and the right arm 34 are illustrated, wherein the PICC 12 is inserted into a vein of the right arm 34 at a venipuncture location 36. The positive (+) input to the monitor 52 is via electrode 38 which is connected to the left side of the chest 40 via electrical lead wire 31. The negative (−) input to the monitor 52 is via electrical lead wire 42 which is connected electrically directly to the proximal end 47 of the stylet 44. The stylet 44 is inserted into the catheter 46.

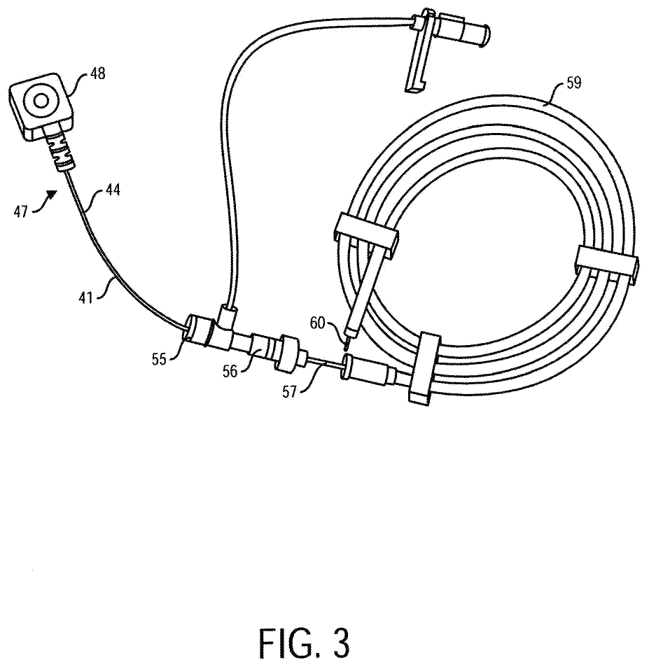

In accordance with the present invention, a Quickly Attached/Quickly Released (QA/QR) electrical connector, such as a male snap connector 48 (FIG. 3), is manufactured to be permanently connected to the proximal end 47 of the stylet 44. The male snap connector 48 can then be quickly connected to the female snap connector 50 which is electrically connected to an ECG/EKG monitor 52 having inputs 37. The stylet 44 is inserted into the catheter 46 and locked in position within the catheter by a screw lock 55 located on the hemostasis 56. After the stylet 44 is used to properly position the distal end of the catheter 46 within the SVC of the patient, the screw lock 55 is released, and the stylet 44 is removed from the catheter 44. The catheter is then secured in place with an IV connector mount 58, such as a microclave or end cap. The ECG lead connectors 48, 50 and 38 of the present invention can be utilized on any ECG monitor, but are preferably used on a small portable EGC monitor 52 to wait for a spike in the P-wave 54 when properly positioning the distal end 15 of the PICC 12 within a patient 10.

The ECG monitor 52 is preferably a small portable model, such as the Wellue Pulsebit™ EX personal ECG/EKG monitor sold by Viatom, based out of Guangdong, China. The Pulsebit™ is designed to wirelessly interface with a sophisticated ECG tracking chart and an analyzing program on a smartphone, such as the iPhone®. Actually, any monitor can be utilized with the present invention as long as the monitor includes input leads for receiving and displaying input signals.

FIG. 3 illustrates a stylet 44 configured in accordance with the present invention. The proximal end 47 of the stylet 44 includes a male snap connector 48 for quickly and easily electrically connecting the stylet 44 to the negative input of an ECG monitor. While the illustrated quick connector 48 shown in FIG. 3 is a male snap connector, any quick electrical connector can also be utilized, such as an alligator clip, plug, etc. The quick connector 48 preferably is permanently secured to the distal end 47 of the stylet 44.

The stylet 44 is shown within a hemostasis valve with a T-port 56 and a dispenser tube 59 housing the stylet 44 before insertion into a catheter. The hemostasis valve 56 includes a screw lock 55 for securing the stylet 44 within the hemostasis valve 56 so as to extend a predetermined length beyond the hemostasis valve 56. This predetermined length 57 ensures the distal end 60 of the stylet 44 is located at the distal end of a catheter, but does not exit the distal end of a catheter within a patient. The hemostasis valve 56 is removed along with the stylet 44.

Also, in accordance with a further embodiment of the present invention, the stylet wire 44 is covered with a vinyl insulation 41, such as nylon, polyvinyl chloride or PTFe (polytetrafluoroethylene), specifically constructed to enable a specific PICC line, called the Hydropicc by Access Vascular, Inc., to function without interference or aspects in the monitoring of the P-wave when using the stylet 44 as a negative contact for an ECG monitor. This vinyl insulation 41 provides an significant improvement when using the stylet 44 as a negative contact as certain PICC lines are constructed mostly of water that carry an electrical charge on the surface of the PICC line, so any non-insulated surface of the stylet causes significant P-wave impedance, resulting in no change in the P-wave, which prevents a technician from properly positioning the distal end of the catheter using an ECG monitor.

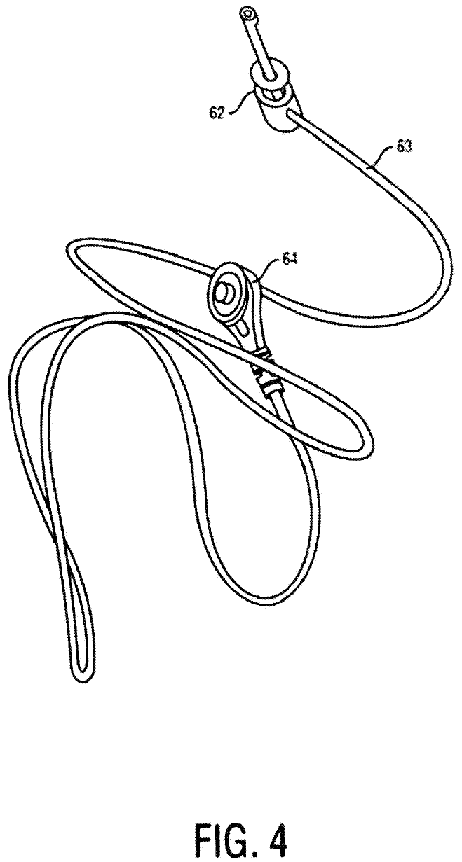

FIG. 4 illustrates an electrical wire lead 63 including a QA/QR electrical connector. Here, the QA/QR electrical connector is a spring engaged hook clip 62 for quickly connecting to and releasing from the proximal end 47 of the stylet 44. This is another embodiment of the present invention that enables a negative lead of an ECG monitor to be quickly connected to the proximal end 47 of a stylet 44 using the spring engaged hook clip connector 62. This provides an alternative to the male snap connector 48 permanently connected to the proximal end 47 of the stylet 44 shown in FIGS. 2 and 3. The opposing end of the electrical lead 63 includes a male snap connector 64 for quickly connecting the electrical lead 63 to the negative input of an ECG monitor.

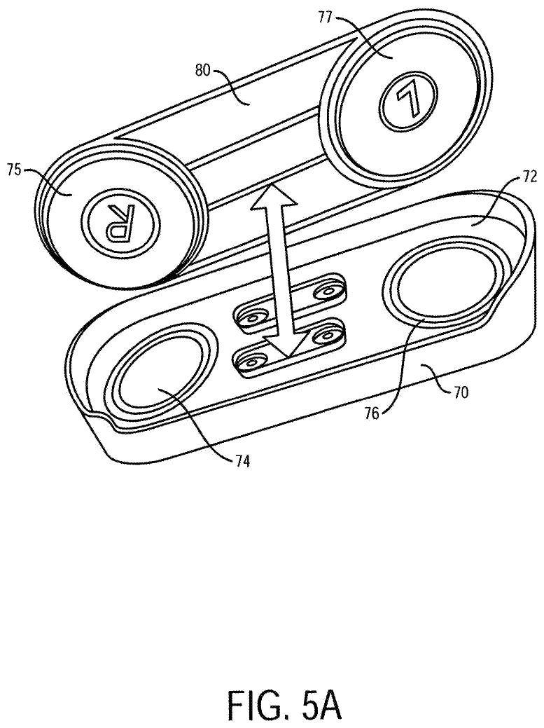

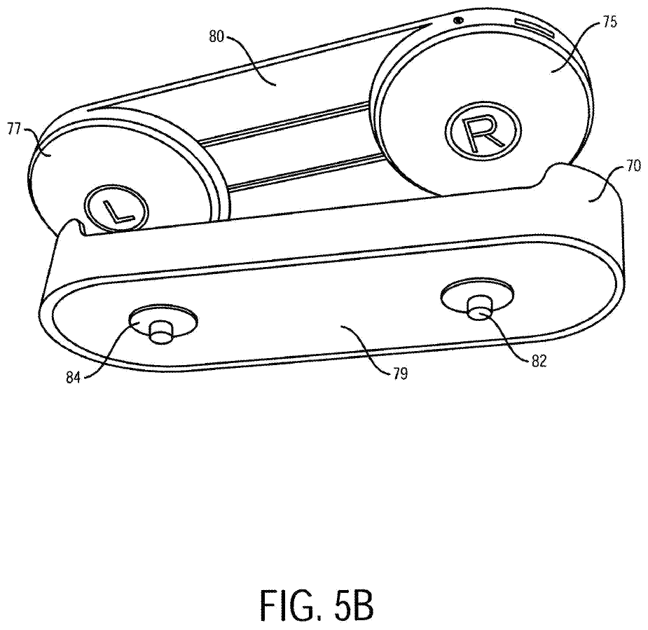

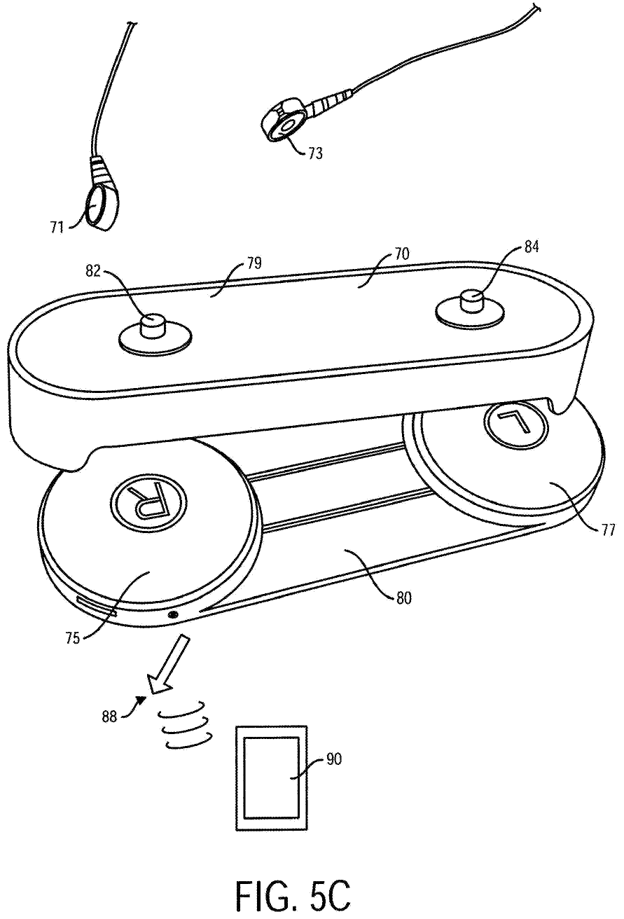

FIG. 5a is a perspective view of the bottom 72 of an ECG lead connector coverplate 70 configured in accordance with the present invention. The coverplate 70 is configured to securely fit over a portable ECG or EKG detection device 80, such as the KardiaMobile monitor manufactured and sold by AliveCor, Inc. or the Wellue DuoEK™ wearable EKG/ECG detection device, which includes Bluetooth capability for interfacing with a sophisticated EKG/ECG monitoring and analyzing application program on a smartphone, such as an iPhone®. The coverplate 70 includes a right (R) electrical contact 74 and a left electrical contact 76 that directly contact the right 75 and left 77 electrical contacts, respectively, of the portable EKG detection device 80. The right and left electrical contacts 75 and 77, respectively, were originally configured as part of the Wellue DuoEK™ to place a finger on each of the right and left hand of a patient.

FIG. 5b illustrates the top 79 of the coverplate 70 which includes knobs or snap button connectors 82 and 84 corresponding to the right 74 and left 76 electrical contacts (FIG. 5a) on the bottom 72 of the coverplate 70.

FIG. 5c illustrates the coverplate 70 about to be snapped onto the electrical contacts 75 (R) and 77 (L) of the EKG detection device 80. The right (R) electrical contact 75 is to be connected to the proximal end 47 of the stylet 44 via the female snap button connector 71. The left (L) electrical contact 77 is to be connected to the left side of the patient 10 via the female snap button connector 73. The female snap button connector 71 is preferable used with the Scout™ lead to connect to the stylet 44. Of course, any type of quick attach and release connector can be used in place of the snap button connectors 71, 73, 82, 84.

The EKG detection device 80 then wirelessly transmits 88 detected EKG data to a smart phone 90 with a display screen to display EKG readings, such as an iPhone®.

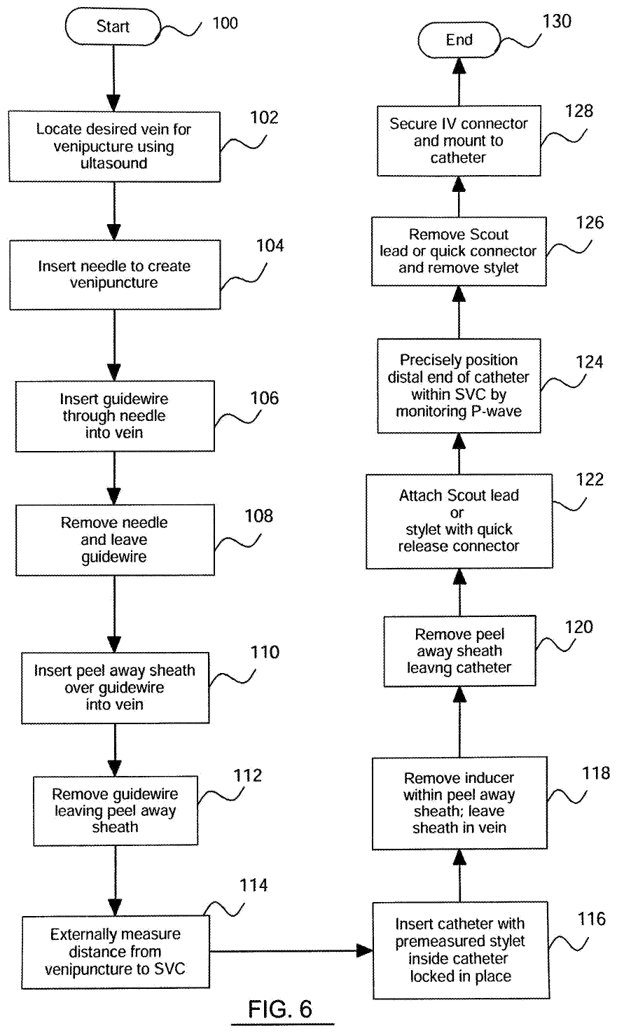

FIG. 6 is a flow chart setting forth steps of the present invention for inserting a PICC line. Beginning at start 100, the first step 102 is to locate a desired vein to insert a needle using ultrasound. Then in step 104 the technician inserts a needle into the desired vein. Next at step 106 a guidewire is inserted though the needle and into the vein, and then the needle is removed leaving the guidewire at step 108. A peel away sheath is then inserted over the guidewire and into the vein in step 110. Next at step 112 the guidewire is removed leaving the peal away sheath. At step 114 the distance from the venipuncture or insertion point of the needle to the SVC is measured. A catheter is then inserted into the peel away sheath with the premeasured stylet corresponding to the distance to the end of the catheter at step 116. The stylet is locked in place inside the catheter so the distal end of the stylet is located at the distal end of the cather, but does not exit the external end of the catheter. Next at step 118 an inducer or dilator is removed from the peel away sheath, leaving the sheath within the vein at the venipuncture. At step 120 the peel away sheath is removed, leaving the catheter within the vein.

In accordance with the present invention, at step 122 the Scout™ lead or a quick release connector is attached to the proximate end of the stylet to create or form a negative (−) connection lead for the ECG. At step 124 the distal end of the catheter is precisely located in the SVC by monitoring the P-wave, wherein the distal end of the stylet is located at and within the distal end of the catheter, thus enabling the ECG to precisely position the distal end of the catheter by monitory the distal end of the stylet. After the distal end of catheter is properly located within the SVC, the Scout™ lead or quick connector disconnected from the stylet and the stylet is removed in step 126. Next in step 128 an IV connector is connected to the proximal end of the catheter and is glued by adhesive to the skin of the patient adjacent to the venipuncture. The process for inserting the PICC in accordance with the present invention is terminated at step 130.

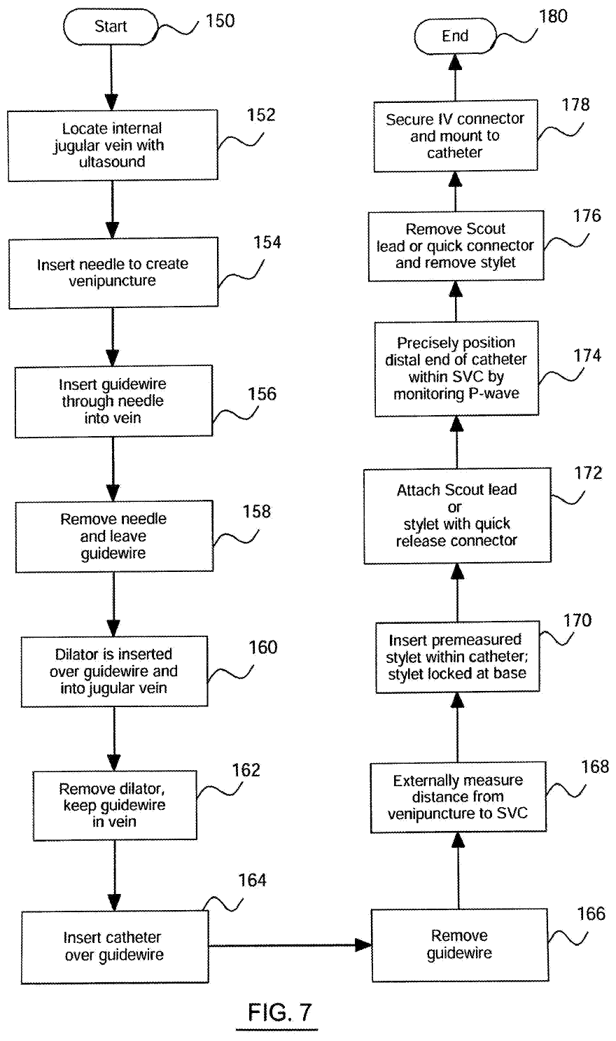

FIG. 7 is a flow chart of the process of inserting a dialysis catheter is accordance with the present invention. Beginning at start 150, the first step 152 is to locate the jugular vein using ultrasound and then insert a needle at step 154. At the next step 156 the technician inserts a guidewire through the needle and into the jugular vein. The needle is then removed leaving the guidewire at step 158. An introducer or dilator is inserted over the guidewire and into the jugular vein at step 160. Next at step 162 the dilator is removed, and the guidewire remains in the jugular vein. At step 164 the catheter is inserted over the guidewire and into the jugular vein, then at step 166 the guidewire is removed leaving the catheter in the jugular vein. At step 168 the distance from the venipuncture or insertion point of the needle to the SVC is externally measured. At step 170 a premeasured stylet corresponding to the distance from the venipuncture to the SVC is inserted into the catheter so at reach the distal end of the catheter, but not exit the distal end of the catheter.

In accordance with the present invention, at step 172 the Scout™ lead or a quick release connector is attached to the proximate end of the stylet to create or form a negative (−) connection lead for the ECG. At step 174 the distal end of the catheter is precisely located in the SVC by monitoring the P-wave, wherein the distal end of the stylet is located at and within the distal end of the catheter, thus enabling the ECG to precisely position the distal end of the catheter by monitory the distal end of the stylet. After the distal end of catheter is properly located within the SVC, the Scout™ lead or quick connector disconnected from the stylet and the stylet is removed in step 176. Next in step 178 an IV connector is connected to the proximal end of the catheter and is glued by adhesive to the skin of the patient adjacent to the venipuncture. The process for inserting the dialysis in the jugular vein in accordance with the present invention is terminated at step 180.

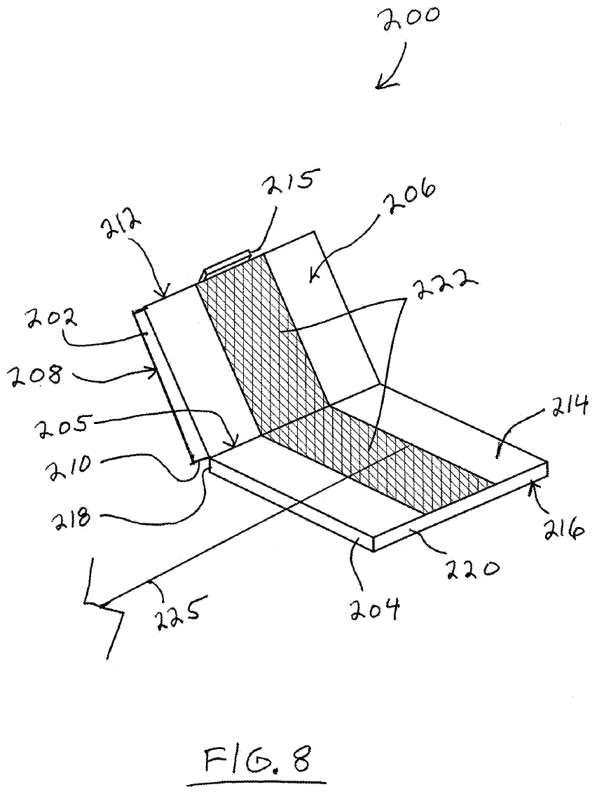

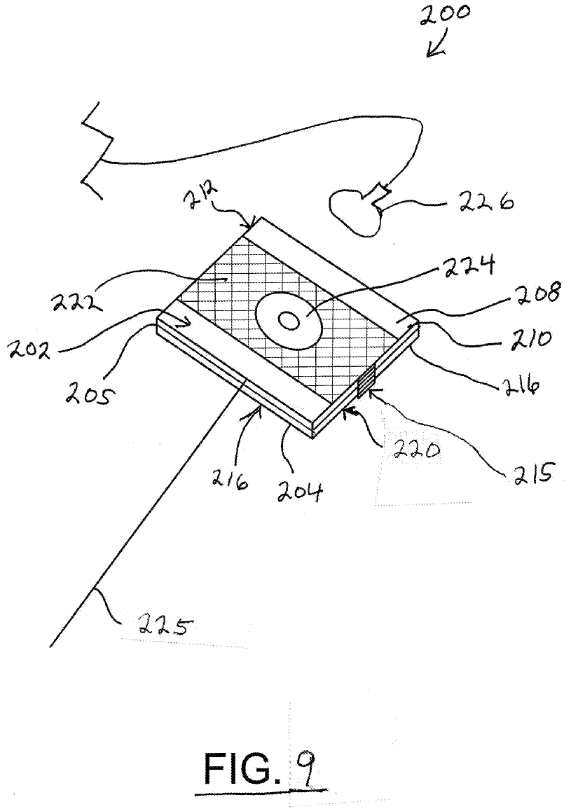

Referring now to FIGS. 8 and 9, illustrated are perspective views of an electrical connector clamp or Scout™ connector clamp 200 configured in accordance with the present invention. FIG. 8 illustrates the connector clamp 200 in the open position and FIG. 9 illustrates the connector clamp 200 in the closed position. The connector camp 200 includes a first plate 202 pivotally connected to a second plate 204 by a hinge 205. The hinge 205 can be a separate component or a crease formed between the first plate 202 and the second plate 204 that preferably is integrally formed out of the same single material, such as stainless steel or any conductive metal or a flexible material such as plastic. The first plate 202 preferably is planar and has a first planar side 206 and a second planar side 208. The first plate 202 further includes a first edge 210 and a second edge 212, wherein a latch 215 is connected to the second edge 212, and the hinge 205 is located on the first edge 210. The second plate 204 has a third planar side 214, a fourth planar side 216, a third edge 218, and a fourth edge 220. The plate 204 preferably is planar, and the third edge 218 is pivotally connected to the first edge 210 of the first plate 202.

An electrically conductive layer 222, such as copper, or a magnetic metal, preferably is attached to the first side 206 of the first plate 202, especially if the first plate 202 is not constructed of an electrically conductive material. The electrically conductive layer 222 can wrap around to the second side 208 of the first plate 202 to electrically connect to the electrical connector 224, wherein the electrical connector 224 is located on the second side 208 of the first plate 202. The electrical connector 224 preferably is a button connector, male or female. The electrical connector 224 can be magnetic and attached to the second side 208 of the first plate 202 by magnetism or other known electrically conductive means, such as soldering. The first plate 202 can be constructed of magnetic material to attach the electrical connector 224 to the first plate 202 by magnetism. The first plate 202 also can be constructed of a conductive material, thus eliminating the need for a conductive layer 222.

The second plate 204 also can include a conductive layer 222 on the third side 214 and the fourth side 216. The latch 215 on the second side 212 of the first plate 202 is configured to latch or grip the fourth edge 220 of the second plate 204 in the closed position, as shown in FIG. 9.

In accordance with the present invention, and as shown in FIG. 8, a wire 225 is to be placed between the first and second plates 202,204 and secured between the first and second plates 202,204 when the plates 202,204 are in the closed position, as shown in FIG. 9. The wire 225 also is electrically connected to the electrical connector 224 when the wire 225 is secured between the first and second plates 202,204 in the closed position. Another matching electrical connector 226, such as a female button connector, can then be connected to the electrical connector 224, thus establishing an electrical connection between the wire 225 and the electrical connector 226.

Assuming the wire 225 is the proximal end 47 of the stylet 44, an electrical connection can be made to an ECG monitor as discussed above.

While specific embodiments have been shown and described to point out fundamental and novel features of the invention as applied to the preferred embodiments, it will be understood that various omissions and substitutions and changes of the form and details of the invention illustrated and in the operation may be done by those skilled in the art, without departing from the spirit of the invention.

1. An electrical connector clamp for attaching to a distal end of a stylet of a Central Venous Access Device (CVAD), comprising:
a first plate having a first side, a second side, a first edge, and a second edge;
a second plate having a third side, a fourth side, a third edge, and a fourth edge, wherein the first edge and the third edge are connected by a hinge that enables the first plate and second plate to pivot relative to each other, and the hinge enables the first side of the first plate and the third side of the second plate to come in contact when in a closed position;
a latch for holding the first side of the first plate against the third side of the second plate in the closed position;
electrically conductive material on the first side of the first plate;
an electrical connector on the second side of the first plate that is electrically connected to the electrically conductive material on the first side of the first plate; and
wherein the first side and third side are to be pivoted together in the closed position around a wire located between the first plate and third plate to provide to an electrical connection between the wire and the electrical connector.

2. The electrical connector clamp of claim 1, further comprising:
electrically conductive material on the third side of the second plate.

3. The electrical connector clamp of claim 1, further comprising:
electrically conductive material on the second side of the first plate that is electrically connected to the electrically conductive material on the first side and the electrical connector.

4. The electrical connector clamp of claim 1, wherein the electrical conductor is magnetically connected to the second side of the first plate.

5. The electrical connector clamp of claim 1, wherein the first plate has a planar configuration.

6. The electrical connector clamp of claim 1, wherein the second plate has a planar configuration.

7. The electrical connector clamp of claim 1, wherein the electrical conductor is male button connector.

8. The electrical connector clamp of claim 1, wherein the first plate and the second plate are constructed of a single material, and the hinge is formed by a crease between the first and second plates.

9. The electrical connector clamp of claim 1, further comprising:
a wire located between the first and second plates in the closed position and electrically connected to the electrical connector.

10. The electronical connector clamp of claim 7, further comprising:
a female button connector electronically attached to the mail button connector.

11. The electronical connector clamp of claim 1, further comprising:
electrically conductive material on the second side of the first plate.

12. The electrical connector clamp of claim 1, wherein the latch is connected to the first plate.

13. The electrical connector clamp of claim 1, further comprising:
a stylet wire located and secured between the first and third plates in the closed position.

14. The electrical connector of clamp claim 1, wherein the electrically conductive material is an electrically conductive metal.

15. An electrical connector clamp for attaching to a distal end of a stylet of a Central Venous Access Device (CVAD), comprising:
a first plate having a first side, a second side, a first edge, and a second edge;
a second plate having a third side, a fourth side, a third edge, and a fourth edge, wherein the first edge and the third edge are connected by a hinge that enables the first and second plates to pivot relative to each other, and the hinge enables the first side of the first plate and the third side of the second plate to come in contact when in a closed position;
a latch for holding the first side of the first plate against the third side of the second plate in the closed position;
the first side of the first plate has an electrically conductive surface;
an electrical connector on the second side of the first plate that is electrically connected to the electrically conductive surface on the first side of the first plate; and
wherein the first side and third side are to be pivoted together in the closed position around a wire located between the first plate and third plate to provide to an electrical connection between the wire and the electrical connector.

16. The electrical connector clamp of claim 15, wherein the first plate is constructed of metal having a conductive surface.

17. The electrical connector clamp of claim 15, wherein the first plate includes magnetic material, and the electrical connector is attached to the first plate by magnetism.

18. The electrical connector clamp of claim 15, wherein the first plate includes metal material, and the electrical connector is magnetic and attached to the first plate by magnetism.

19. The electrical connector clamp of claim 15, wherein the first plate and the second plate are constructed of unitary plastic, and the hinge is formed by a crease between the first plate and the second plate, and the first side of the first plate includes metal plating to create a conductive surface.

20. The electrical connector clamp of claim 15, wherein the first plate and the second plate are separate components.
